(19) United States
(12) Patent Application Publication
(10) Pub. No.: US 2002/0012998 A1
(43) Pub. Date: Jan. 31, 2002

(54) Cationic liposomes

(76) Inventors: Igor Gonda, San Francisco, CA (US); Rimona Margalit, Tel Aviv (IL)

Correspondence Address: Paula Borden, BOZICEVIC, FIELD & FRANCIS LLP, 200 Middlefield Road, Suite 200, Menlo Park, CA, 94025, US

(21) Appl. No.: 09/823,256

(22) Filed: Mar. 29, 2001

Related U.S. Application Data
(60) Provisional application No. 60/193,062, filed on Mar. 29, 2000.

(51) Int. Cl. A61K 9/127

(52) U.S. Cl. 435/458; 424/450; 514/44

(57) ABSTRACT

The cationic liposomal formulations of the present invention provide nucleic acid and gene product delivery devices having a glycosaminoglycan covalently attached to the liposome surface. The glycosaminoglycan can be any glycosaminoglycan, including but not limited to hyaluronic acid, the chondroitin sulfates, keratan sulfate, chitin and heparin. Preferably, the glycosaminoglycan is hyaluronic acid. The present invention also provides methods of preparing the nucleic acid-liposome formulations.

### CROSS REFERENCE

This application claims the benefit of U.S. Provisional Patent Application Ser. No. 60/193,062, filed Mar. 29, 2000, which application is incorporated herein by reference in its entirety.

### TECHNICAL FIELD

The field of the invention is generally directed toward the use of cationic liposomes for complexing with nucleic acid. Such complexes may be used for introduction of nucleic acids and/or gene products into cells.

### BACKGROUND OF THE INVENTION

A number of methods have been used for delivery and expression of foreign genes in vitro and in vivo. These include chemical methods (calcium phosphate precipitation, DEAE-dextran, polybrene, neutral or anionic liposomes, cationic liposomes and targeted polylysine conjugates etc.), physical methods (microinjection, electroporation and biobalistics) and biological methods (viral vectors) (Felgner (1993) J. Liposome Res., 3:3-16).

Practically speaking, an ideal gene delivery vector should have the following characteristics: (1) it should protect and deliver DNA into cells efficiently, preferably with specificity toward a particular cell type; (2) it should be non-toxic and non-immunogenic; and (3) it should be easy to produce in large quantity. To date, none of the vector systems can meet all these requirements.

Adenovirus, for example, is a highly efficient vector for gene transfer and can transiently infect cells of different types. Engineered adenovirus is believed to be relatively safe for the host (Rosenfeld et al. (1992) Cell, 68:143-155; Engelhardt et al. (1994) Proc. Natl. Acad. Sci. USA, 91:6196-6200). Compared with other recombinant viral vectors, adenovirus is relatively easy to produce in large quantity. However, recent preclinical and clinical trials have raised serious concerns about its immunogenicity. Treatment related inflammation, production of neutralizing antibodies and virus specific cytotoxic T lymphocyte (CTL) response in the host may prevent this viral vector from being used at high doses or administered repeatedly (Crystal et al. (1994) Nature Genetics, 8:42-51).

Retrovirus and adeno-associated virus (AAV) mediate efficient and stable transfection to dividing and possibly nondividing cells (Miller (1990) Hum Gene Ther, 1:5; Kotin (1994) Hum Gene Ther, 5:793-801). However, relatively low viral titers have been the major technical limitation for both systems.

The safety concerns and the difficulty of obtaining a large quantity of the recombinant viral vector have prompted the search for efficient, nonimmunogenic or poorly immunogenic and easy-to-prepare nonviral vector systems. Among them, cationic liposomes and targeted polylysine conjugates are the most promising (Felgner et al. (1991) Nature, 349:351-352; Curiel et al. (1991) Proc Natl Acad Sci USA, 88:8850-8854).

A number of methods of liposomal nucleic acid delivery have been described. U.S. Pat. No. 5,908,635 describes liposomal preparations of cationic lipopolyamines and a neutral lipid for gene therapy. U.S. Pat. No. 5,908,777 describes a method of making lipidic vectors for nucleic acid delivery by condensing a nucleic acid in a nucleic acid/polycationic complex, and then combining that complex with an anionic lipidic preparation to form a lipidic vector. U.S. Pat. No. 5,783,566 describes a method for controlling transfection efficiency mediated by complexes of cationic species and genetic material by adjusting the amount of membrane-associated proteoglycans and optionally adjusting the plasma concentration of glycosaminoglycans. U.S. Pat. No. 5,827,703 describes the use of lipid carriers comprising cationic lipids and cholesterol for DNA expression cassettes.

Although many liposome-based methods exist, the above described cationic liposome-DNA constructs used for gene therapy have a number of critical deficiencies, including toxicity, immunogenicity, macrophage phagocytosis, and lack of targeting ability. Moreover, when used in pulmonary delivery, such constructs are susceptible to mucociliary clearance.

Thus, a need exists to provide improved liposomes for delivery of nucleic acids and gene products. This need and others are addressed by the instant invention.

### SUMMARY OF THE INVENTION

The cationic liposomal formulations of the present invention provide nucleic acid and gene product delivery devices characterized by (1) bioadhesivity to target cells (2) reduced immunogenicity, e.g., resistance to macrophage phagocytosis, and (3) a “masking” of the cationic lipids at the surface of the liposome, resulting in a change in the liposome's nature so that it is recognized as partial or completely self rather than as a foreign entity. The bioadhesive cationic liposomes of the invention have a glycosaminoglycan covalently attached to the liposome surface. The glycosaminoglycan can be any glycosaminoglycan, including but not limited to hyaluronic acid, the chondroitin sulfates, keratan sulfate, chitin and heparin. Preferably, the glycosaminoglycan is hyaluronic acid. The present invention also provides methods of preparing the nucleic acid-liposome formulations.

The bioadhesive cationic liposome delivery devices of the invention provide a means for introducing a nucleic acid and/or a gene product into a cell, thereby providing a method for administering the product to the cell for a variety of purposes. These delivery devices can be administered as a pharmaceutical formulation, i.e. with an excipient carrier. The invention provides a pharmaceutical liposomal formulation for the delivery of nucleic acids useful in systemic administration and provides long-term expression of a given gene in dividing cells.

Although the bioadhesive cationic liposomes can be administered via any conventional route, in a preferred embodiment they are aerosolized and delivered via a pulmonary route.

In one embodiment, the cationic liposomes have at least two biomolecules covalently attached to the cationic liposome, a glycosaminoglycan and another molecule that directs the liposome to a particular cell type, e.g., a ligand (e.g., a hormone) that selectively binds to a transmembrane receptor such as HER-2 or FGFR.

It is an object of the present invention to provide bioadhesive macromolecules for the introduction of a nucleic acid or a gene product into a cell.

It is another object of the invention to provide a liposomal formulation that is not immunogenic when administered to cells in vivo.

It is yet another object of the invention to provide a liposomal formulation with increased concentration and residence at a target cell surface.

It is an advantage that the bioadhesive cationic liposomes exhibit bioadhesive properties that facilitate introduction of nucleic acids into cells.

It is yet another advantage that the bioadhesive cationic liposomes are resistant to macrophage phagocytosis.

It is yet another advantage that the presence of the glycosaminoglycans at the liposome surface masks the cationic lipids at the particle surface.

These and other objects, advantages and features of the present invention will become apparent to those persons skilled in the art upon reading the details of the presently described invention.

### BRIEF DESCRIPTION OF THE DRAWING

FIG. 1 is a graph depicting the effect of saline concentration on emitted dose for DNA/lipid formulations.

### DETAILED DESCRIPTION OF THE PREFERRED EMBODIMENTS

Before the present method of generating the liposomes of the invention, delivery of such for treatment of various medical conditions, devices and formulations used in connection with such are described, it is to be understood that this invention is not limited to the particular methodology, devices and formulations described, as such may, of course, vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “and,” and “the” include plural referents unless the context clearly dictates otherwise. Thus, for example, reference to “a formulation” includes mixtures of different formulations, reference to “an analog” refers to one or mixtures of analogs, and reference to “the method of treatment” includes reference to equivalent steps and methods known to those skilled in the art, and so forth.

Unless defined otherwise, all technical and scientific terms used herein have the same meaning as commonly understood by one of ordinary skill in the art to which this invention belongs. Although any methods, devices and materials similar or equivalent to those described herein can be used in the practice or testing of the invention, the preferred methods, devices and materials are now described. All publications mentioned herein are incorporated herein by reference in their entirety for the purpose of describing and disclosing devices, formulations and methodologies which are described in the publication and which might be used in connection with the presently described invention.

The terms “nucleic acid” or “polynucleotide” as used herein are considered interchangeable unless otherwise indicated, and encompass DNA, RNA or a mixture of DNA and RNA. Nucleic acids according to the present invention may also include any strand structure, e.g., single-, double- or triple-stranded polynucleotide structures or mixtures thereof. Also, the nucleic acids may comprise a linear or circular structures, e.g., plasmids, phagemids, cosmids, etc.

The term “gene product” as used herein refers an oligopeptide, peptide, or protein generated from a nucleic acid introduced into or to a cell using the methods of the present invention.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. In one embodiment, “treatment” as used herein covers any treatment of a disease in a mammal, particularly a human, and includes:

(a) preventing the disease from occurring in a subject which may be predisposed to the disease but has not yet been diagnosed as having it;

(b) inhibiting the disease, i.e., arresting its development; or

(c) relieving the disease, i.e., causing regression of the disease. The invention is directed toward treating patients for biological processes involving cell migration such as inflammation and is particularly directed toward treating cancer cell metastasis. In another embodiment, the term “treatment” as used herein covers any use for inhibiting or enhancing a normal biological process, such as oocyte fertilization.

The term “isolated” means the protein is removed from its natural surroundings. However, some of the components found with it may continue to be with an “isolated” protein. Thus, an “isolated protein” is not as it appears in nature but may be substantially less than 100% pure protein.

The term “glycosaminoglycan” as used herein refers to a macromolecule comprised of carbohydrate, including both known or as yet unidentified glycosaminoglycans. The glycosaminoglycans for use in the present invention may vary in size and be either sulfated or non-sulfated. The glycosaminoglycans which may be targeted using the inhibitors and methods of the invention include, but are not limited to, hyaluronic acid, the chondroitin sulfates, keratan sulfate, chitin and heparin.

By “binds specifically” is meant high avidity and/or high affinity binding of a liposome to specific cell surface molecule, e.g. the liposomal glycosaminoglycan binds to a cell surface molecule such as CD44. Binding of the liposome to a specific cell surface molecule is preferably stronger than binding of the same liposome to other proteins on the cell or to related but different proteins on other cells, particularly other cells which may be present in the same region as the target cells. Liposomes that bind specifically to cells may also be able to bind to other cell surface molecules at a weaker, yet detectable, level (e.g., 10% or less of the binding shown the target cell surface molecule). Such weak binding, or background binding, is readily discernible from the specific of interest, e.g. by use of appropriate controls.

General Aspects of the Invention

The present invention provides efficient methodologies to effectively bind various bioadhesive molecules to liposomes in order to increase the delivery efficacy of nucleic acids contained within and/or associated with the liposomes to specific cell populations. The liposome delivery systems of the invention is created by the binding of glycosaminoglycans to liposomal surfaces thereby forming bioadhesive liposomes. The bioadhesive liposomes of the present invention have specificity for and the ability to adhere to the designated target area and once adhered to a target will provide for sustained release of the liposome's therapeutic contents.

The improved process of the present invention includes adding a bioadhesive molecule to a liposome; adding a crosslinking reagent to the mixture of the liposome and bioadhesive molecule; and, allowing the mixture to incubate for a period of time to form the bioadhesive liposome. By modifying regular liposomes through covalent bonding of certain bioadhesive molecule to the liposomal surface, the bioadhesive molecules can be utilized as an adhesive or glue to attach and retain the modified liposome onto a target area despite cellular and fluid dynamics. The product formed comprises a plurality of liposome-crosslinker-bioadhesive molecule complexes which can contain or associate with a therapeutic agent comprising a nucleic acid. The nucleic acid can either prevent expression of an endogenous protein (e.g., an antisense oligonucleotide) or will encode and express a therapeutic gene product.

The bioadhesive cationic liposomes of the present invention can be used as pharmacological agents that are targeted to cells having cell surface molecules, such as CD44, that bind with specificity to HA. These bioadhesive cationic liposomes have numerous advantages for introducing nucleic acids and their resulting gene products into cells in vivo.

First, these molecules exhibit bioadhesive properties that improve transfection efficacy and allow dose reduction. The glycosaminoglycan facilitates binding of the liposome to the cell membrane, facilitating cell entry. This results not only in increase in liposome concentration at the target cells, it also results in improved residence time at the target cells. When the liposome is delivered via a pulmonary route, the residence time is also increased due to mucociliary clearance.

Second, the bioadhesive cationic liposomes display an improved resistance to immune response, as they are resistant to macrophage phagocytosis. This results in a reduction in macrophage-mediated clearance compared to other liposomal delivery devices. In addition, the early immune responses against these liposomes are inhibited, resulting in a reduction in immunogenicity and toxicity. This reduced immunogenicity and toxicity are dose dependent.

Third, the presence of the glycosaminoglycans at the liposome surface masks the cationic lipids at the particle surface, and the liposomes are thus recognized in vivo as partially self or self rather than foreign matter.

These and other advantages of the liposome delivery devices of the present invention overcome many of the shortcomings of conventional non-viral nucleic acid delivery methods. In addition, the delivery devices do not have many of the shortcomings associated with physical or viral means of introduction of nucleic acids and gene products into cells in vivo.

### NUCLEIC ACID CONSTRUCTS FOR DELIVERY

The nucleic acids which can be encapsulated or otherwise associated with the liposome-crosslinker-bioadhesive molecule according to the present method may comprise sense or antisense polynucleotides. For example, antisense oligonucleotides used may selectively inhibit the expression of target DNA's. For example, antisense oligonucleotides may be encapsulated which are complementary to viral sequences and utilized for antiviral treatments, e.g., hepatitis, AIDS viral infection, papillomavirus infection, etc. The use of antisense oligonucleotides for genetic therapy has been reported in the literature. See Stein and Chang,(1993) Science 261: 1004. Also, ribozymal RNA's may be encapsulated and used to study gene expression or for genetic therapy.

In one embodiment, the present invention provides for the efficient encapsulation of high molecular weight polynucleotide molecules. As used herein, “high molecular weight” polynucleotide refers to a polynucleotide molecule that comprises at least one coding sequence that can be transcribed when the polynucleotide is introduced into a host cell. This transcription can produce an mRNA molecule that can then be translated to produce a polypeptide or protein, or it can produce an antisense RNA molecule. Transcription of the coding sequence of the HMW polynucleotide is preferably under the control of cis-acting regulatory elements, such as enhancer sequences, operator sequences and the like, and the polynucleotide also contains a ribosome binding site, an initiation codon and transcription termination and polyadenylation signals. The definition of HMW polynucleotides as used herein is, therefore, generally understood to mean polynucleotides that contain such regulatory elements. The HMW polynucleotide may also contain other elements such as origins of replication as are commonly found on polynucleotides used for transfection.

The present invention provides for the efficient encapsulation of large vectors, including those which have operably integrated therein sequences that permit stable, episomal maintenance and those which encode multigene cassettes. This is significant, in the case of episomal constructs, because integration of the desired nucleic acid into the host cell's genome may have a negative impact on the transfection process. For multigene cassettes, it also is important as coordinate regulation of the encoded genes can be more easily achieved.

The nucleic acids which may be encapsulated according to the present method may range in size from as small as about 500 bases to about 50 kilobases. In a preferred embodiment, the encapsulated nucleic acids will comprise DNA's ranging from about 1.0 to 25 kB and, preferably, from about 5 to about 18 kB.

In a preferred embodiment, the encapsulated nucleic acids will comprise an episomal element, e.g., a plasmid which contains one or more genes which are to be expressed in target cells. An episomal element containing an origin of replication that is recognized by the replication functions of the host cell will be stably maintained in the cell as an extrachromosomal element, thereby allowing stable expression of genes encoded on the element. In general, these genes will cause the target cell to produce a heterologous expression product, or acquire an altered phenotype. If the episomal element does not contain an origin or replication that is recognized by the host cell, the expression product will be produced only transiently.

As discussed supra, the nucleic acid encapsulated may comprise DNA, RNA or a mixture thereof, and may comprise linear or circular structures. Also, the encapsulated nucleic acids may be single or multi-stranded and may comprise sense or antisense nucleic acid sequences. In the preferred embodiment, the nucleic acids will comprise DNA constructs having a size ranging from about 5 to about 18 kilobases. In general, such DNA constructs will contain a gene or genes which are to be expressed in the targeted cells. The DNA construct also preferably will contain suitable regulatory sequences which provide for the expression of these genes, in addition to sequences that provide for these DNA constructs to autonomously replicate in target cells if necessary, and also suitable selectable markers, e.g., antibiotic resistance markers. In general, these genes will be expressed under the control of regulatable promoters.

In the most preferred embodiments, the DNA constructs will contain a gene or genes which produce a therapeutic or desired gene product. Examples of such gene products include, but are not limited to, therapeutic lymphokines, cytokines, hormones, cell adhesion molecules, enzymes or enzyme inhibitors, receptors, ion channels, transcription factors, protein kinases, protein phosphatases, and cellular antigens for generating an immune response in a host. Alternatively the DNA constructs will contain suicide genes, tumor suppressor genes, genes encoding antisense RNAs, or genes that induce or prevent cellular apoptosis.

Examples of lymphokines and cytokines that can be encoded by the liposomally-encapsulated DNA constructs of the invention include but are not limited to platelet-derived growth factor, epidermal growth factor, interleukins 1-14, granulocyte colony stimulating factor, granulocyte-macrophage colony stimulating factor, tumor necrosis factor, leukemia inhibitory factor, amphiregulin, angiogenin, betacellulin, calcitonin, ciliary neurotrophic factor, brain-derived neurotrophic factor, neurotrophins 3 and 4, nerve growth factor, colony stimulating factor-1, endothelial cell growth factor, erythropoietin, acidic and basic fibroblast growth factor, hepatocyte growth factor, heparin binding EGF-like growth factor, insulin, insulin-like growth factors I and II, interferons alpha., beta., and gamma., keratinocyte growth factor, macrophage inflammatory protein alpha. and .beta., midkine, oncostatin M, RANTES, stem cell factor, transforming growth factors alpha. and .beta., and vascular endothelial growth factor. Examples of cell adhesion molecules include integrins, cadherins, selecting, and adhesion molecules of the immunoglobulin superfamily, such as VCAM, ICAM, PECAM, and NCAM. Examples of tumor suppressor genes include p53, DCC, Rb, and MTS1. Those of skill in the art will recognize that other genes can also be used in the invention.

In addition, the DNA construct will contain regulatory elements that can control replication of the construct within the cell, as well as transcription and translation of genes encoded on the construct. For use in in vivo delivery of nucleic acids, it is sometimes useful for these regulatory elements to be tissue specific. The term “tissue-specific promoter” or “tissue-specific transcriptional regulatory sequence” or indicates a transcriptional regulatory sequence, promoter and/or enhancer that is induced selectively or at a higher level in cells of the target tissue than in other cells. For example, tumor cell-specific promoters include promoters that are induced selectively or at a higher level in a particular cell type or a tumor cell. Tissue specific promoters are known in the art. Examples include: the alpha-actin promoter (Shani (1986), Mol. Cell. Biol., 6:2624); the elastase promoter (Swift et al.(1984), Cell, 38:639); the alpha-fetoprotein promoter (Krumlauf et al.(1985), Nature, 319:224-226); the beta-globin promoter, (Townes et al.(1985), EMBO J., 4:1715); the human growth hormone promoter (Behringer et al.(1988), Genes Dev., 2:453); the insulin promoter (Selden et al.(1986), Nature, 321:545) and a prostate-specific promoter (Allison et al.(1989), Mol Cell. Biol., 9:2254).

### CATIONIC LIPOSOME PREPARATION

In general, a variety of methods are available for preparing liposomes as described in, e.g., Szoka et al., Ann. Rev. Biophys. Bioeng. 9:467 (1980), U.S. Pat. Nos. 4,235,871, 4,501,728, 4,837,028, the text Liposomes, Marc J. Ostro, ed., Marcel Dekker, Inc., New York, 1983, Chapter 1, and Hope, et al., Chem. Phys. Lip. 40:89 (1986), all of which are incorporated herein by reference. One method produces multilamellar vesicles of heterogeneous sizes. In this method, the vesicle-forming lipids are dissolved with the nucleic acids to be encapsulated in a suitable organic solvent or solvent system and dried under vacuum or an inert gas to form a thin lipid film. If desired, the film may be redissolved in a suitable solvent, such as tertiary butanol, and then lyophilized to form a more homogeneous lipid mixture which is in a more easily hydrated powder-like form. This film is covered with an aqueous buffered solution and allowed to hydrate, typically over a 15-60 minute period with agitation. The size distribution of the resulting multilamellar vesicles can be shifted toward smaller sizes by hydrating the lipids under more vigorous agitation conditions or by adding solubilizing detergents such as deoxycholate.

For example, lipofectin (Gibco BRL, Gaithersburg, Md.) has been successfully used for the transfection of various cell lines in vitro (Felgner et al.(1987), Proc. Natl. Acad. Sci. U.S.A., 84:7413-7417) and for systemic gene expression after intravenous delivery into adult mice (Zhu et al.(1993), Science, 261:209-211). Lipofectin is formed with the cationic lipid DOTMA, N-[1-(2,3-dioleyloxy) propyl]-N,N,N-trimethylammonium chloride, and DOPE, dioleylphosphatidyl ethanolamine at a 1:1 molar ratio. The liposomes prepared with this formulation are thought to spontaneously interact with DNA through the electrostatic interaction of the negative charges of the nucleic acids and the positive charges at the surface of the cationic liposomes. This DNA/liposomal complex fuses with tissue culture cells and facilitates the delivery of functional DNA into the cells (Felgner et al., supra).

Behr et al.(1989), Proc. Natl. Acad. Sci. U.S.A., 86:6982-6986) and Barthel et al. (1993), Cell Biol, 12:553-560) have recently reported the use of a lipopolyamine (DOGS, Spermine-5-carboxy-glycinediotadecylamide) to transfer DNA to cultured cells. Lipopolyamines are synthesized from a natural polyamine spermine chemically linked to a lipid. For example, DOGS is made from spermine and dioctadecylamidoglycine (Behr et al., supra). DOGS spontaneously condense DNA on a cationic lipid layer and result in the formation of nucleolipidic particles. This lipospermine-coated DNA shows high transfection efficiency (Barthel et al., supra).

Cationic liposomes containing multivalent cationic lipid usually show better transfection activities than those containing lipids (Behr et al., (1989) Proc Natl Acad Sci USA, 86:6982-6986; Hawley-Nelson et al. (1993) Focus, 15:73-79). For example, LipofectAMINE (GIBCO BRL, Gaithersburg, Md., USA) is consistently more active in transfection than Lipofectin (GIBCO BRL) (Hawley-Nelson et al., supra).

All cationic lipid molecules contain four different functional domains: a positively charged head group(s), a spacer of varying length, a linker bond and a hydrophobic anchor. The head group of most known cationic lipids contains a simple or multiple amine group with different degrees of substitution, with one exception being an amidine group (Ruysschaert et al., (1994), Biochem Biophys Res Commun, 179:280-285). The amine groups range from primary amine to quaternary ammonium with substitution of methyl or hydroxyethyl groups. In some cases several different types of amino groups coexist in a single cationic lipid (dioctadecyldimethylammonium chloride (DOGS) and 2,3-dioleoyloxy-N-(2(sperminecarboxamido)-ethyl)-N,N-dimethyl-1-propanamimium trifluoroacetate (DOSPA)) (Behr et al., supra; Hawley-Nelson et al., supra). The number of charged groups varies from monovalent to multivalent (Felgner et al. (1987), Proc Natl Acad Sci USA, 84:7413-7417; Felgner et al.(1994), J. Biol. Chem., 269:1550-1561; Behr et al., supra; Farhood et al. (1992), Biochim. Biophys. Acta., 1111:239-246; Gao et al. (1991), Biochim. Biophys. Res. Commun., 179:280-285; Zhou et al. (1991), Biochim. Biophys. Acta., 1065:8-14; Rose et al. (1991), Biotechniques, 10:520-525; Hawley-Nelson et al., supra; Ruysschaert et al., supra; Ito et al. (1990), Biochem. Intl., 22:235-241; Leventis et al. (1990), Biochem. Biophys. Acta., 1023:124-132; Guo et al. (1993), J. Liposome Res., 3:51-70; Akao et al. (1994), Biochem. Mol. Biol. Intl., 34:915-920). The head group of a cationic lipid is responsible for interactions between liposome and DNA, and between liposome-DNA complex and cell membrane or other components of the cell. The interaction is vital for the transfection activity and may contribute to the toxicity of the treatment. The importance of choosing a particular type of head group has been demonstrated in our studies with cationic cholesterol derivatives (Farhood et al., supra). Cationic cholesterol compounds with tertiary amino groups show more superior transfection activity and less toxicity to the treated cells than their quaternary ammonium counterparts (Farhood et al., supra). In another study by Felgner's group, hydroxyethyl substituted derivatives of N-(2,3-(dioleoyloxy)propyl)-N,N,N-trimethyl ammonium chloride (DOTMA) show improved transfection activity over the parent compound (Felgner et al. (1994), supra). It has been hypothesized that the additional hydroxyethyl group enhances membrane hydration and helps to stabilize the liposomal bilayer structure. Cationfic lipids with multivalent head groups bind to DNA and form complexes that are more compact than those formed between DNA and monovalent cationic lipids, which may at least partially contribute to the high transfection activities of the multivalent cationic lipids-counting liposomes (Behr et al., supra; Zhou et al., supra; Hawley-Nelson et al., supra).

For some cationic lipid systems, the spacer arm appears to be less critical for the transfection activity. Examples include the cationic lipid dimethyldioctadecylammonium bromide (DDAB), which contains no spacer, and cationic lipids such as 1,2-dioleoyloxy-3-(trimethylammonio) propane (DOTAP), DOTMA and their derivatives, which contain only a one-atom spacer. Increasing the length of spacer to five to eight atoms did not provide the analogs with better transfection activity than DOTAP (Leventis et al., supra). The studies from our group on the series of functional cholesterol derivatives also indicated that the length of spacer arm can be varied between three and six atoms (Farhood et al., supra). On the other hand, a spacer arm is a crucial factor for the transfection activity of certain cationic lipids. For example, a spacer arm is important for a lipid-anchored spermine head group to bind DNA in the minor groove (Leoffler et al. (1990) J Neurochem, 54:1812-1815), and for high transfection activity of the lipid (Leoffler et al., supra; Remy et al. (1994) Bioconjugate Chem, 5:647-654). Liposomes composed of cationic lipid with longer spacers show enhanced interaction with the surface of mucosal tissue (Guo et al., supra). From the study of a series of cationic glutamate diesters by Ito et al., it seems that cationic lipids containing long spacers (11 atoms) are less active than the ones with shorter spacers (two to six atoms) (Ito et al., supra).

The linker bond is an important parameter which determines the chemical stability and biodegradability of a cationic lipid. Cationic lipids such as DOTMA and DDAB contain ether or C-N bonds. Such stable linkers provide excellent chemical stability, but they are not likely to be biodegradable in the cell. The safety of cationic lipids containing these stable bonds, for use in humans, is thus questionable, especially for repeated administrations. Cationic lipids with ester bonds are more biodegradable and therefore have less cytotoxicity (Farhood et al., supra; Leventis et al., supra; Guo et al., supra). However, cationic lipids with ester bonds as linkers are generally not chemically stable. For example, liposome composed of DOPE and a cationic cholesterol derivative with an ester bond showed a half-life of about one day at 4 C, which makes it difficult to use practicality (Farhood et al., supra). Therefore several cationic lipids were designed to contain stable but biodegradable bonds such as amide and carbamoyl bonds (Farhood et al., supra; Gao et al, supra; Ito et al., supra).

Two types of hydrophobic anchors have been used in cationic lipids, one is a pair of aliphatic chains, and the other is a cholesterol ring. The fatty chains are generally monounsaturated (oleoyl, C18) or shorter saturated (C14 or C12) which offer sufficient membrane fluidity and good lipid mixing within the bilayer at physiological temperature (Feigner et al. (1987), supra; Felgner et al. (1994), supra; Ito et al., supra; Leventis et al., supra). Felgner et al. have recently shown that as the length of the saturated aliphatic chain was increased from 14 to 16 and 18 carbon atoms, the transfection activity of the resulting cationic lipids progressively declined (Felgner et al. (1994), supra). On the other hand, lipospermines containing either two palmityl chains (C16, saturated), or two oleoyl chains (C18 monounsaturated) show high transfection activity (Remy et al., supra), suggesting that the physical property of the lipid anchor is less important for multivalent cationic lipids that monovalent cationic lipids.

A cholesterol ring can also serve as a useful hydrophobic anchor because it exists naturally and derivatives with various functional groups are readily available (Farhood et al., supra; Gao et al., supra; Leventis et al., supra; Guo et al., supra). Cholesterol is known for its capability of keeping lipids in good packing order and thus offers rigidity to the bilayer membrane. Liposomes containing a cationic cholesterol derivative are more stable in the presence of interfering substances such as ions in the culture media (Leventis et al., supra), or a negatively charged mucus surface (Guo et al., supra). On the other hand, cationic liposomes with a cationic cholesterol derivative display equivalent, or better fusion activity than that of cationic liposomes containing cationic lipids with double aliphatic chains (Leventis et al., supra). A number of the cholesterol based cationic lipids have been reported (Farhood et al., supra; Gao et al., supra; Leventis et al, supra; Guo et al., supra). Cholesterol derivatives with quaternary ammonium head groups showed weak but detectable in vitro transfection activity (Leventis et al., supra). Results with this group showed that tertiary but not quaternary derivatives of cholesterol are active cationic lipids (Farhood et al., supra). Another derivative with a -alanine linked to the cholesterol ring via an ester bond showed good activity (Guo et al., supra). We have reported a simple procedure to synthesize a tertiary amino derivative with a carbamoyl bond (3(N-(N′,N′-dimethylaminoethane) carbamoyl)-cholesterol (DC-chol)), which provides excellent stability and transfection efficiency as well as potential biodegradability, and hence reduced toxicity compared with the widely used cationic lipid with an ether bond (Gao et al., supra).

Examples of lipids suitable for use in the invention include, e.g., known vesicle or liposome forming compounds such as phosphatidylcholine, both naturally occurring and synthetically prepared, phosphatidic acid, lysophosphatidylcholine, phosphatidylserine, phosphatidyl ethanolamine, sphingolipids, phosphatidylglycerol, sphingomyelin, cardiolipin, glycolipids, gangliosides, and cerebrosides such as soybean phospholipids. Other suitable lipids include steroids, cholesterol, aliphatic amines such as long chain aliphatic amines and carboxylic acids, long chain sulfates and phosphates, diacetyl phosphates, butylated hydroxy toluene, tocopherol, retinol and isoprenoid compounds which may confer desired properties to the formed liposomes.

Also, synthetic phospholipids containing either altered aliphatic portions such as hydroxyl groups, branched carbon chains, cyclo derivatives, aromatic derivatives, ethers, amides, polyunsaturated derivatives, halogenated derivatives or altered hydrophilic portions containing carbohydrate, glycol, phosphate, phosphamide, quaternary amines, sulfate, sulfonyl, carboxy, amine, sulphydryl, imidazole groups and combinations of such groups can be either substituted or intermixed with the above-mentioned lipids which may be used in the present invention. Lipids suitable for use in preparing liposomes are well known in the literature, and are described, e.g., in U.S. Pat. Nos. 4,201,767; 4,235,877; 4,241,046; 4,261,975; and 4,394,448, all of which are incorporated by reference in their entireties.

An active cationic liposome formulation is usually small, unilamellar liposomes prepared by sonication or microfluidization (Felgner et al. (1987) supra). Occasionally, multilamellar liposomes prepared by simple vortex (Felgner et al. (1994), supra), or dilution of lipid solution from ethanol solvent (Behr et al (1989), supra), are also active. Cationic liposomes normally contain a cationic amphiphile and a neutral ‘helper’ lipid, dioleoylphosphatidylethanolamine (DOPE). DOPE is required for non-bilayer forming cationic lipids to form stable cationic liposomes; these include cationic cholesterol derivatives (Farhood et al. (1992), supra; Gao et al. (1991), supra), lipopolylysine (Zhou et al. (1991), supra), and some double-chain cationic surfactants (Rose et al. (1991), supra). Most double-chain cationic lipids can form liposomes by themselves, or form liposomes as a mixture with DOPE or other lipids.

The present invention embraces the use of any lipid or combination which provides the desired encapsulation efficiency which, in general, will be at least about 25%, relative to the initial amount of nucleic acid used for encapsulation. In a preferred embodiment the encapsulation efficiency will be preferably at least 50%, more preferably 70%, and still more preferably will be in the range from about 70% to about 90%.

Following liposome preparation, the liposomes may be sized to achieve a desired size range and relatively narrow distribution of liposome sizes. A size range of about 0.2-0.4 microns allows the liposome suspension to be sterilized by filtration through a conventional filter, typically a 0.22 micron filter. The filter sterilization method can be carried out on a high through-put basis if the liposomes have been sized down to about 0.2-0.4 microns.

Several techniques are available for sizing liposomes to a desired size. One sizing method is described in U.S. Pat. No. 4,737,323, incorporated herein by reference. Sonicating a liposome suspension either by bath or probe sonication produces a progressive size reduction down to small unilamellar vesicles less than about 0.05 microns in size. Homogenization is another method which relies on shearing energy to fragment large liposomes into smaller ones. In a typical homogenization procedure, multilamellar vesicles are recirculated through a standard emulsion homogenizer until selected liposome sizes, typically between about 0.1 and 0.5 microns, are observed. In both methods, the particle size distribution can be monitored by conventional laser-beam particle size discrimination.

Extrusion of liposome through a small-pore polycarbonate membrane or an asymmetric ceramic membrane is also an effective method for reducing liposome sizes to a relatively well-defined size distribution. Typically, the suspension is cycled through the membrane one or more times until the desired liposome size distribution is achieved. The liposomes may be extruded through successively smaller-pore membranes, to achieve a gradual reduction in liposome size. For use in the present inventions, liposomes having a size of from about 0.05 microns to about 0.15 microns are preferred.

After the nucleic acid-containing liposomes are produced, the composition preferably will be treated to remove the free nucleic acids. This may be effected by any suitable method which does not adversely affect the liposomes, for example, by washing the liposomes in a suitable solution, e.g., phosphate buffered saline, followed by centrifugation. Nucleic acid-containing liposomes produced in this manner may be used immediately or may be stored under favorable conditions, e.g., at about 4° C.

After the liposomes are produced, the encapsulation efficiency may be ascertained by known methods. For example, a DNA sample containing radiolabeled plasmid DNA constructs can be employed. This permits determination of the relative amounts of liposome-contained and free radioactivity. The subject method reliably provides for nucleic acid entrapment efficiencies ranging from at least 25 to 50%, and more typically about 70 to 90% based upon the initial amount of nucleic acid contained in the sample, e.g., a DNA plasmid containing sample.

### Preparation of Cationic Liposomes with a Covalently Attached Bioadhesive Molecule

Bioadhesive molecules are bound covalently to discrete sites on the liposome surfaces. The number and surface density of these sites will be dictated by the liposome formulation and the liposome type. The liposomal surfaces may also have sites for noncovalent association. Covalent binding is essential as noncovalent binding might result in dissociation of bioadhesive molecules from the liposomes at the site of administration since the liposomes and the bioadhesive counterparts of the target site (the bioadhesive matter) compete for the bioadhesive molecules. Such dissociation would reverse the administered modified liposomes into regular, non-modified liposomes, thereby defeating the purpose of administration of the modified liposomes.

To form covalent conjugates of bioadhesive molecules and liposomes, crosslinking reagents have been studied for effectiveness and biocompatibility. Crosslinking reagents include glutaraldehyde (GAD), bifunctional oxirane (OXR), ethylene glycol diglycidyl ether (EGDE), and a water soluble carbodiimide, preferably 1-ethyl-3-(3-dimethylaminopropyl) carbodiimide (EDC). Examples of such crosslinkers and methos of use are described in U.S. Pat. No. 5,603,872, issued Feb. 18, 1997, which is incorporated herein by reference. Through the complex chemistry of crosslinking, linkage of the amine residues of the bioadhesive molecule and liposomes is established.

The present invention provides attached glycosaminoglycans as bioadhesive molecules of the surface of cationic liposome delivery devices. The role of glycosaminoglycans, and particularly hyaluronic acid (HA), in varying physiological processes make them attractive candidates for bioadhesive molecules. Glycosaminoglycans, and particularly HA, are known to mediate cellular interactions that involve binding and entry into a cell. For example, HA is involved in infection of mammalian cells by the Human Immunodeficiency Virus (HIV), since HIV is known to bind to HA upon infection. Both HA and monoclonal antibodies to its receptor CD44 were found to inhibit HIV infection of monocytes by monocytotropic HIV. M. C. Levesque and B. F. Haynes, J. Immunol 156:1557-65 (1996). Moreover, glycosaminoglycans have been found to be nearly non-antigenic, and very few antibodies that recognize glycosaminoglycans have been isolated.

HA is a disaccharide that exists in vivo as a high molecular weight linear polysaccharide. HA is found in mammals predominantly in connective tissues, skin, cartilage, and in synovial fluid, and is also the main constituent of the vitreous of the eye. In connective tissue, the water of hydration associated with HA creates spaces between tissues, thus creating an environment conducive to cell movement and proliferation. HA plays a key role in biological phenomena associated with cell motility including rapid development, regeneration, repair, embryogenesis, embryological development, wound healing, angiogenesis, and tumorigenesis (Toole, Cell Biol. Extracell. Matrix, Hay (ed), Plenum Press, New York, 1384-1386 (1991); Bertrand et al. Int. J. Cancer 52:1-6 (1992); Knudson et al, FASEB J. 7:1233-1241 (1993)). HA levels have been shown to correlate with tumor aggressiveness (Ozello et al., Cancer Res. 20:600-604 (1960); Takeuchi et al., Cancer Res. 36:2133-2139 (1976); Kimata et al., Cancer Res. 43:1347-1354 (1983)), and can be indicative of the invasive properties of tumor cells. M. M. Knupfer et al., Anticancer Res 18:353-6 (1998).

Several groups have identified CD44 as a receptor for HA (See, e.g., Aruffo et al., Cell, 61:1303 (1990); Lesley et al., Exp. Cell. Res., 187:224 (1990); Miyake et al., J. Exp. Med., 172:69 (1990); Culty et al., J. Cell Biol., 111:2765 (1990)). CD44 is a family of cell-surface glycoproteins generated by alternative splicing and post-translational modification Various isoforms of CD44 are expressed by many cell types, such as T cells and B cells, granulocytes, monocytes, macrophages, and DC, including LC. In 1996, HA-CD44 interaction was reported to mediate rolling of leukocytes over endothelial cells, one of the numerous processes that take place in leukocyte homing (See, e.g., DeGrendele et al., J. Exp. Med., 111:2765 (1996); Clark et al., J. Cell Biol, 134:1075 (1996)). HA-CD44 interaction is now known to mediate many physiological and pathological events, including leukocyte homing, tumor metastasis, tissue development, hematopoiesis, cytokine production, T cell activation, and apoptosis.

Other molecules may also serve as bioadhesive molecules with the glycosaminoglycans, and can serve to bind the liposomes to specific cell populations. Such factors bind specifically to cell surface molecules on target cells. Bioadhesive molecules include, but are not limited to growth factors, such as EGF, FGF, VEGF, insulin, etc., cytokines, such as the interleukins, including from IL-1to IL-18, particularly IL-2, IL-4, IL-12 and IL-18; interferons, including IFN-α, IFN-β, IFN-γ; chemokines; TNF-α, TGFβ, and the like.

### Uses of the Liposomes of the Invention

The bioadhesive liposomal encapsulated nucleic acids will have many different potential uses, as will be apparent to one skilled in the art upon reading the present disclosure. For example, the bioadhesive liposomal encapsulated nucleic acids can be used to produce cells or animals which express a defective gene or genes. The resulting cells or animals may be used as in vitro or in vivo models for assessing the efficacy of potential therapeutic agents.

A further utility for the liposomally encapsulated nucleic acids of the invention is for introducing into cells DNA or constructs that encode a therapeutic product or prevent transcription of an endogenous product. The therapeutic product can be, for example, an antisense RNA or ribozyme RNA molecule, or it can be a therapeutic protein. A “therapeutic protein” as used herein refers to a peptide, polypeptide, or protein that, when confers a therapeutic benefit to a host when administered to the host, or when it is expressed in cells of the host. The nucleic acid delivery can be in vivo, in which the liposomally encapsulated DNA constructs are introduced directly into a host animal, preferably a human, or can be ex vivo, in which isolated cells are first transfected with the liposomally encapsulated DNA constructs, and are then reintroduced into a host animal. Ex vivo nucleic acid delivery in humans is described in U.S. Pat. No. 5,399,346, which is hereby incorporated by reference in its entirety. See also Tolstoshev (1993), Annu. Rev. Pharmacol. Toxicol., 33:573-96, for a general review of nucleic acid delivery, which is also incorporated herein by reference in its entirety.

### Pharmaceutical Formulations of the Liposomes of the Invention

The presently described bioadhesive cationic liposomes may be administered to a subject by virtually any means used to administer conventional antibiotics. A variety of delivery systems are well known in the art for delivering bioactive compounds to an animal. These systems include, but are not limited to, intravenous or intra-muscular or intra-tracheal injection, nasal spray, aerosols for inhalation, and oral or suppository administration. The specific delivery system used depends on the location of the area to be treated, and it is well within the skill of one in the art to determine the location and to select an appropriate delivery system. In a preferred embodiment, the liposomal compositions are delivered via pulmonary introduction, and more preferably the bioadhesive cationic liposomes are administered to a patient in an aerosol inhalation device.

In a particularly preferred embodiment, the formulations of the invention are preferably administered to a patient using a portable, hand-held, battery-powered device, such as the AERx device (Aradigm, Hayward, Calif.). Alternatively, the formulations of the instant invention could be carried out using a mechanical (non-electronic) device. Specific devices that may be used are disclosed in more detail in U.S. Pat. No. 5,544,646, issued Aug. 13, 1996 and U.S. Pat. No. 5,404,871, issued Apr. 11, 1995, which both are incorporated herein by reference.

An aerosol may be created by forcing drug through pores of a membrane which pores have a size in the range of about 0.25 to 6 microns. When the pores have this size the particles which escape through the pores to create the aerosol will have a diameter in the range of 0.5 to 12 microns. Drug particles may be released with an air flow intended to keep the particles within this size range. The creation of small particles may be facilitated by the use of the vibration device which provides a vibration frequency in the range of about 800 to about 4000 kilohertz. Those skilled in the art will recognize that some adjustments can be made in the parameters such as the size of the pores from which drug is released, vibration frequency, pressure, and other parameters based on the density and viscosity of the formulation keeping in mind that the object is to provide aerosolized particles having a diameter in the range of about 0.5 to 12 microns.

Formulations of the invention can include bioadhesive cationic liposomes combination with an amount of alveolar surfactant protein effective to enhance the transport of the liposomes across the pulmonary surface and into the circulatory system of the patient. Exemplary formulations are disclosed within U.S. Pat. No. 5,006,343, issued Apr. 9, 1991, which is incorporated herein by reference to disclose liposomes and formulations of liposomes used in intrapulmonary delivery.

The liposome formulation may be a low viscosity liquid formulation. The viscosity of the drug by itself or in combination with a carrier must be sufficiently low so that the formulation can be forced out of openings to form an aerosol, e.g., using 20 to 200 psi to form an aerosol preferably having a particle size in the range of about 0.5 to 12 microns.

In an embodiment, a low boiling point, highly volatile propellant is combined with the liposomes of the invention and a pharmaceutically acceptable excipient. The liposomes can be provided as a suspension or dry powder in the propellant, or, in another embodiment, the liposomes are dissolved in solution within the propellant. Both of these formulations may be readily included within a container which has a valve as its only opening. Since the propellant is highly volatile, i.e., has a low boiling point, the contents of the container will be under pressure.

In accordance with another formulation, the bioadhesive cationic liposomes are provided as a dry powder by itself, and in accordance with still another formulation, the bioadhesive cationic liposomes are provided in a solution formulation. The dry powder could be directly inhaled by allowing inhalation only at the same measured inspiratory flow rate and inspiratory volume for each delivery. However, the powder is preferably dissolved in an aqueous solvent to create a solution which is moved through a porous membrane to create an aerosol for inhalation.

Any formulation which makes it possible to produce aerosolized forms of bioadhesive cationic liposomes which can be inhaled and delivered to a patient via the intrapulmonary route can be used in connection with the present invention. Specific information regarding formulations which can be used in connection with aerosolized delivery devices are described within Remington's Pharmaceutical Sciences, A. R. Gennaro editor (latest edition) Mack Publishing Company. Regarding insulin formulations, it is also useful to note Sciarra et al. (1976), Journal of Pharmaceutical Sciences, Vol. 65, No. 4. When low boiling point propellants are used, the propellants are held within a pressurized canister of the device and maintained in a liquid state. When the valve is actuated, the propellant is released and forces the active ingredient from the canister along with the propellant. The propellant will “flash” upon exposure to the surrounding atmosphere, i.e., the propellant immediately evaporates. The flashing occurs so rapidly that it is essentially pure active ingredient which is actually delivered to the lungs of the patient.

In preparing the compositions in oral dosage form, any of the usual pharmaceutical media may be employed, such as, for example, water, glycols, oils, alcohols, flavoring agents, preservatives, coloring agents and the like in the case of oral liquid preparations (such as, for example, suspensions, elixirs, and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated and enteric-coated by standard techniques.

For parenteral application by injection, preparations may comprise a pharmaceutically acceptable form of the liposomes in an appropriate solution. Injectable suspensions may also be prepared using appropriate liquid carriers, suspending agents, agents for adjusting the isotonicity, preserving agents, and the like. Actual methods for preparing parenterally administrable compositions and adjustments necessary for administration to subjects will be known or apparent to those skilled in the art and are described in more detail in, for example, Remington's Pharmaceutical Science, 15th Ed., Mack Publishing Company, Easton, Pa. (1980), which is incorporated herein by reference.

For topical administration, the carrier may take a wide variety of forms depending on the preparation, which may be a cream, dressing, gel, lotion, ointment, or liquid.

Suppositories are prepared by mixing the liposome with a lipid vehicle such as theobroma oil, cacao butter, glycerin, gelatin, or polyoxyethylene glycols.

An effective amount of composition to be employed therapeutically will depend, for example, upon the therapeutic objectives, the route of administration, and the condition of the patient. Accordingly, it will be necessary for the clinician to titer the dosage and modify the route of administration as required to obtain the optimal therapeutic effect.

Regardless of the form of the drug formulation, it is preferable to create liposome particles for inhalation in the range of about 0.5 to 12 microns. By creating liposome particles which have a relatively narrow range of size, it is possible to farther increase the efficiency of the drug delivery system and improve the repeatability of the dosing. Thus, it is preferable that the particles not only have a size in the range of 0.5 to 12 microns but that the mean particle size be within a narrow range so that 80% or more of the particles being delivered to a patient have a particle diameter which is within ±20% of the average particle size, preferably ±10% and more preferably ±5% of the average particle size.

Additionally, liposomes according to the present invention may be administered in vivo in combination with other medicaments suitable for use in treating a particular disorder. For example, if the liposomes contain a “suicide gene” which renders targeted cells susceptible to a particular drug, it may be desirable to coadminister liposomes in with the drug. The drug may be, but need not be, liposomally encapsulated.

Dosing

A proposed daily dosage of active compound for the treatment of man is about 0.5 mg DNA/kg to 4 mg DNA/kg, which may be conveniently administered in one or two doses. The precise dose employed will of course depend on the age and conditions of the patient and on the route of administration. Thus a suitable dose for administration by inhalation is about 0.5 mg DNA/kg to 2 mg DNA/kg, for oral administration is about 2 mg DNA/kg to 5 mg DNA/kg, for parenteral administration is about 2 mg DNA/kg to 4 mg DNA/kg.

These liposomes may be used for both in vitro or in vivo transfection of nucleic acids into targeted cells. The targeted cells can be any cell whose cellular membrane is comprised of a lipid bilayer, and in general will comprise eukaryotic cells, and preferably mammalian cells, more preferably murine or human cells.

If transfection is effected in vitro, a suitable amount of the subject liposomes will be added to a cell culture medium containing the targeted cells. A suitable amount of the liposome composition may range from about 0.12 to 1.2 mg of liposome per 10.⁶ cells, or from about 0.1 to about 10 μg of encapsulated DNA per 10.⁶ cells. Those of skill in the art will realize, however, that this amount can vary, depending upon factors such as the lability of the particular targeted cell, its resistance to transfection, whether the liposomes contain smaller or larger nucleic acids, the activity of the particular gene, and the desired level of gene expression.

The resulting liposomally-transfected cells may be used for various applications. For example, the cells may be used to express a polypeptide encoded by the incorporated nucleic acids, e.g., a desired mammalian gene product. Also, if the incorporated nucleic acids result in the cells expressing a particular genetic defect, the cells may be used as models for studying the efficacy of proposed therapies for the particular genetic defect. Alternatively, if in vitro liposomal transfection results in the incorporation of genes which compensate for some genetic defect, or which encode a moiety such as an antisense RNA, ribozyme, or therapeutic protein, these cells may be administered to a host in need of genetic therapy. See U.S. Pat. No. 5,399,346.

If the nucleic acid-containing liposomes are to be used in vivo, they are administered to a host in need of such treatment. Another variation on in vivo use is for the generation of genetic defects, e.g., transgenic or “knock-out” mice which are useful in the study of disease. An example of treatment in a patient is when a DNA construct encoding human leukocyte antigen B7 (HLAB7) is encapsulated in a liposome as described supra and injected directly into the tumor lesions of a patient suffering from cutaneous melanoma, as described in Nabel et al. (1993), Proc. Natl. Acad. Sci., 90:11307, which is hereby incorporated by reference in its entirety. The HLAB7 stimulates the host immune response against the melanoma cells.

Generally, an in vivo liposomal dosage will range from about 0.2 to 20 mg/kg of body weight, and preferably from about 2 mg to 10 mg/kg of body weight. The amount will, of course, depend on the particular genetic defect, the type of nucleic acid encapsulated, the desired level of gene expression, the amount of nucleic acid contained in the liposomes, and other factors as discussed supra.

The efficiency of in vivo or in vitro transfection may be measured by standard methods. See Sambrook et al. (1989), Molecular Cloning: a Laboratory Manual, Second Edition, Cold Spring Harbor Laboratory, Cold Spring Harbor, N.Y. For example, the expression of genes encoded on a liposomally encapsulated DNA construct transfected into cells in vitro can be studied by Northern blotting or RNA PCR to measure production of RNA transcripts, and by Western blotting, immunoprecipitation, and in situ immunohistochemistry to detect and measure protein production. Integration of the DNA into the host cell chromosome can be determined by PCR or by Southern blotting. The same methods are used to determine whether tissue treated in vivo contains transfected genes, or is expressing gene products of the transfected genes. This is preferably carried out on a biopsy sample of the tissue of interest.

### EXAMPLES

The following examples are put forth so as to provide those of ordinary skill in the art with a complete disclosure and description of how to make and use the subject invention, and are not intended to limit the scope of what is regarded as the invention.

Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### Example 1

### Preparation of DNA Encapsulated Cationic Liposome

Liposomes composed of DOTMA, N-[1-(2,3-dioleyloxy) propyl]-N,N,N-trimethylammonium chloride, and DOPE, dioleylphosphatidyl ethanolamine at a 1:1 molar ratio DOPE/CHEMS are prepared. 50 mg total lipids is first dissolved in dry chloroform and then dried into a thin film in a round-bottomed flask. Next, the lipid is suspended in 2.5 ml deionized H₂O by vortexing. The suspension then is adjusted to pH 8 and sonicated in a bath-type sonicator for 5 minutes. The resulting liposomes are sized by light scattering and sterilized by filtration through a 0.45 μm filter. 36 μg poly-L-lysine in 400 μL deionized H₂O is rapidly mixed with 48 μg plasmid DNA in 400 μL deionized H₂O, at DNA/polylysine weight ratio 1:0.75. Aliquots of the resulting DNA/polylysine complex are then rapidly mixed with various amounts of the cationic liposomes in equal volumes of deionized H₂O. The plasmid DNA and polylysine formed condensed complexes when rapidly mixed. These complexes are stable when the overall charge is either positive or negative.

DNA/polylysine (1:0.75) complex becomes spontaneously encapsulated when rapidly mixed with the DOTMA, N-[1-(2,3-dioleyloxy) propyl]-N,N,N-trimethylammonium chloride, and DOPE, dioleylphosphatidyl ethanolamine liposomes. The size of the DNA-containing liposome is dependent on the charge ratio between the DNA/polylysine complex and the cationic liposomes.

### Example 2

### Covalent Attachment of Bioadhesive Molecule

An aqueous solutions of HA is pre-activated by incubation with water-soluble carbodiimide, EDC. The components are mixed to yield a preparation system of HA and EDC each at final concentrations of 1.7 mg/ml. The pH of the preparation system was adjusted to 3 by titration with 1N HCl. The preparation system is incubated for 3 hours at 37° C. with stirring.

After the pre-incubation period, the samples as produced in Example 1 are added and followed by the addition of a 0.1M borate buffer at pH 8.5. The liposome mixture is incubated at 37° C. in a shaker bath for 24 hours. Removal of excess unbound HA and reagents was by ultracentrifugation and washings.

### Example 3

### Emitted Dose of DNA/lipid Formulation from AERx Aerosolization Device Increases with Electrolyte Concentration

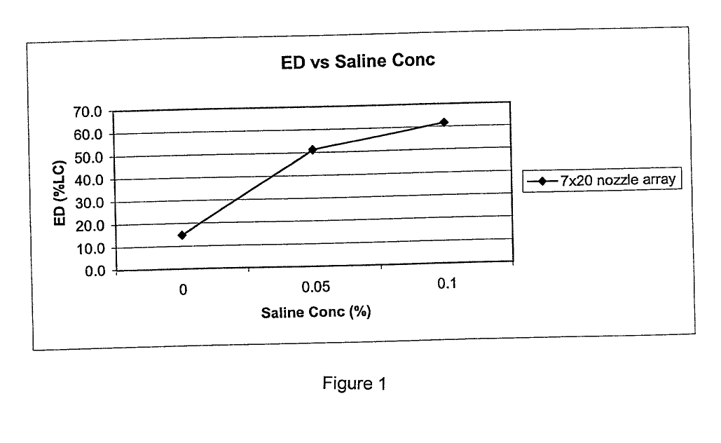

An AERx aerosolization device was used to test emitted doses of DNA/lipid formulations of various salt concentrations. The AERx aerosolization device has a 7×20 nozzle array, 60 LPM, SDP (single dose platform) clamp. DNA/lipid formulations having saline concentrations of 0,0.05, and 0.1% were tested. The results are shown in FIG. 1. The results indicate that the emitted dose of the DNA/lipid formulation increases with increasing salt concentration. ED: emitted dose; %LC: % label claim.

The instant invention is shown and described herein in what is considered to be the most practical, and preferred embodiments. It is recognized, however, that departures may be made therefrom, which are within the scope of the invention, and that obvious modifications will occur to one skilled in the art upon reading this disclosure.

What is claimed is:

1. A composition for transfecting a mammalian cell with a nucleic acid, comprising:
a nucleic acid;
a cationic lipid composition comprised of cationic lipids, which form a liposome; and
a glycosaminoglycan;
wherein the glycosaminoglycan is covalently associated with the surface of the liposome, and further wherein the nucleic acid is characterized as having an activity selected from the group consisting of (a) an ability to hybridize to and inhibit expression of an endogenous nucleic acid and (b) an ability to be transcribed into an active gene product.

2. The composition of claim 1 wherein the cationic lipids are a polyvalent cationic lipid.

3. The composition of claim 1, wherein the glycosaminoglycan is selected from the group consisting of: chondroitin sulfates, keratan sulfate, chitin and heparin.

4. The composition of claim 1, wherein the glycosaminoglycan is hyaluronic acid.

5. The composition of claim I wherein said cationic lipid composition further comprises neutral lipids, and wherein said cationic lipid composition further comprises an alveolar surfactant protein.

6. The composition of claim 1, further comprising a crosslinker connecting the cationic lipid to the glycosaminoglycan.

7. The composition of claim 1, further comprising a second molecule that binds to a second cell surface molecule, wherein said second molecule is covalently attached to the surface of the liposome, and wherein the second molecule is a ligand that specifically binds to a cell surface receptor.

8. A method of transfecting a mammalian cell comprising the steps of:
administering to a mammalian cell a composition comprising a nucleic acid, cationic lipids, and a glycosaminoglycan characterized by an ability to selectively bind to a mammalian cell surface; and
allowing the nucleic acid to enter the cell.

9. The method of claim 8, wherein the nucleic acid encodes a gene product.

10. The method of claim 8, wherein said nucleic acid is an antisense oligonucleotide.

11. The method of claim 8, wherein said nucleic acid is a high molecular weight polynucleotide comprising at least one coding region.

12. A method for making a composition for transfecting a mammalian cell with a nucleic acid which comprises the steps of:
combining a cationic lipid composition comprising a polyvalent cationic lipid and a nucleic acid;
forming a cationic liposome or vesicle from said cationic lipid composition to encapsulate said nucleic acid; and
covalently attaching a glycosaminoglycan to the surface of said cationic liposome.

13. A transfection composition made by the method of claim 12.

14. A method for delivering a nucleic acid to a mammalian cell, said method comprising contacting said cell with a composition of claim 1.

15. The method of claim 14, wherein the nucleic acid is introduced into a mammalian cell in vivo.

16. The method of claim 15, wherein the cell is a human cell.

17. The method of claim 15, wherein the cell is a lung cell, and wherein the composition is administered to said cell via pulmonary delivery of the composition to a subject.

18. The method of claim 17, wherein the composition is aerosolized prior to administration to said lung cell.

19. The method of claim 15, wherein the glycosaminoglycan of the composition is HA and wherein the mammalian cell comprises a cell surface molecule that binds specifically to HA.

20. The method of claim 19, wherein the cell surface molecule is CD44.
